(19) United States
(12) Patent Application Publication
(10) Pub. No.: US 2014/0111630 A1
(43) Pub. Date: Apr. 24, 2014

(54) SYSTEMS AND METHODS FOR IRIS DETECTION AND GAZE ESTIMATION

(71) Applicants: BERNARDO R. PIRES, Pittsburgh, PA (US); TAKEO KANADE, Pittsburgh, PA (US); AKIHIRO TSUKADA, Kanagawa-ken (JP)

(72) Inventors: BERNARDO R. PIRES, Pittsburgh, PA (US); TAKEO KANADE, Pittsburgh, PA (US); AKIHIRO TSUKADA, Kanagawa-ken (JP)

(21) Appl. No.: 13/965,791

(22) Filed: Aug. 13, 2013

Related U.S. Application Data
(60) Provisional application No. 61/742,583, filed on Aug. 14, 2012.

(51) Int. Cl. A61B 3/113; G06K 9/00; A61B 5/00

(52) U.S. Cl. CPC A61B 3/113; A61B 5/6803; G06K 9/00604; G06K 9/0061
USPC 348/78

(57) ABSTRACT

A method for estimating a direction of a subject's gaze includes providing headgear to be worn by the subject, the headgear including an imaging system in operative connection therewith, imaging at least one eyeball using the imaging system over time using light in the visible spectrum, using light in the visible spectrum to determine the position of the iris on a model of the eyeball, and estimating the direction of the subject's gaze from the determined position of the iris on the model of the eyeball.

### CROSS-REFERENCE TO RELATED APPLICATIONS

This application claims benefit of U.S. Provisional Patent Application Ser. No. 61/742,583, filed Aug. 14, 2012, the disclosure of which is incorporated herein by reference.

### GOVERNMENTAL INTEREST

This invention was made with government support under grant no. CC1029549 awarded by the National Science Foundation, grant no. ECHO540865 awarded by the National Science Foundation, and grant no N00014-10-1-0934 awarded by the Office of Naval Research Multi disciplinary University Research Initiative. The government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof The disclosure of all references cited herein are incorporated by reference.

Eye tracking and gaze estimation are key technologies in helping to understand the intent of the user in may situations, including but not limited to impairment detection (e.g. Of drivers and pilots); disease diagnosis and monitoring; measurement of cognitive processes; assessment, modification and control of advertising; assessment of web usability and user interfaces; and use as a controller of computers, video games, and other devices and machinery. In some embodiments of gaze detection systems, wearable devices are used to monitor eye movements, which are in turn correlated with outside images or scenes to determine the focus of the subject's attention. Traditional gaze estimation methods based on active sources, such as intra-red spectrum imaging, may not be desirable for use over extended periods of time, which is required in wearable systems. Moreover, active intra-red systems may not be desirable for use in situations where there can be interference from other sources (for example, from the sun).

### SUMMARY

In one aspect, a method for estimating a direction of a subject's gaze includes providing headgear to be worn by the subject, the headgear including an imaging system in operative connection therewith, imaging at least one eyeball using the imaging system over time using light in the visible spectrum, using light in the visible spectrum to determine the position of the iris on a model of the eyeball, and estimating the direction of the subject's gaze from the determined position of the iris on the model of the eyeball. The imaging system may, for example, include a camera sensitive to light in the visual spectrum.

In a number of embodiments, at least one parameter of the model of the eyeball is determined by determining at least one corner of the eye. The position of the iris on a model of the eyeball may, for example, be determined by detecting at least a portion of an edge of the iris on the model of the eyeball. In a number of embodiments, the iris is modeled as a circle, the eyeball is modeled as a sphere, and the iris is modeled as a circle movable over the surface of the sphere.

In a number of embodiments, the method further includes mapping an image from the imaging system to a θ, φ plane wherein θ is a polar angle about an axis zₑ emanating from the center of the sphere modeling the eyeball, φ is an azimuth angle in an xₑ, yₑ plane which passes through the center of the sphere modeling the eyeball and is orthogonal to the zₑ axis, and determining the position of the iris on the θ, φ plane by detection of the circle modeling the iris on the θ, φ plane. The center of the sphere modeling the eyeball, the radius of the sphere modeling the eyeball and an orientation of xₑ, yₑ plane relative to the imaging system may, for example, be determined by determining the corners of the eye. The corners of the eye may, for example, define aye axis of the sphere modeling the eyeball.

In a number of embodiments, an initial estimate of the position of the iris on the θ, φ plane is made. The initial estimate of the position of the iris on the θ, φ plane may, for example, be made using a Though Transform. A further estimation of the position of the iris on the θ, φ plane may, for example, be made using a circle fitting algorithm that is different from an algorithm used in the initial estimate. Gaze may be estimated using a model that relates the position of the iris on the θ, φ plane to the gaze.

In a number of embodiments, the method further includes imaging a second eyeball using the imaging system over time using light in the visible spectrum, determining the position of the iris of the second eyeball on a sphere modeling the second eyeball by (i) mapping an image from the imaging system to a θ′, φ′ plane wherein θ′ is the polar angle about an axis zₑ′ emanating from the center of the sphere modeling the second eyeball, φ′ is the azimuth angle in an xₑ′, yₑ,′ plane which passes through the center of the sphere modeling the second eyeball and is orthogonal to the zₑ′ axis, and (iii) detecting a circle modeling the iris of the second eyeball on the θ′, φ′ plane, and estimating the direction of the subject's gaze from the determined position of the iris on the eyeball and the second eyeball.

In a number of embodiments, only ambient light is used to image the eyeball. In other embodiments, a source of light may be provided.

In a number of embodiments, the method further includes tracking a position of the eye relative to the imaging system to account for movement of the position of the eye relative to the imaging system in determining the subject's gaze. As described above, a position of the corners of the eye are may be used to determine a position of the eye relative to the imaging system.

The method may further include providing an outside imaging system in operative connection with the headgear, imaging an outside environment using an outside imaging system over time, and relating the subject's gaze to an image of the outside environment. The method may further include calibrating by having the subject gaze at an object in the outside environment having a known position.

In another aspect, a method for estimating a direction of a subject's gaze includes providing headgear to be worn by the subject, the headgear including an imaging system in operative connection therewith, imaging at least one eyeball using the imaging system over time, modeling the eyeball as a sphere, determining the position of the iris on the sphere modeling the eyeball by (i) mapping an image from the imaging system to a θ, φ plane wherein θ is a polar angle about an axis zₑ emanating from the center of the sphere modeling the eyeball, φ is an azimuth angle in an xₑ, yₑ plane which passes through the center of the sphere modeling the eyeball and is orthogonal to the zₑ axis, and (iii) detecting a circle modeling the iris on the θ, φ plane; and estimating the direction of the subject's gaze from the determined position of the iris on the eyeball. In a number of embodiments, the imaging system includes a camera sensitive to light in the visual spectrum.

The position of the iris on the sphere modeling the eyeball may, for example, be determined by detecting at least a portion of an edge of the iris on the sphere modeling the eyeball. The method may further include determining at least one parameter of the sphere modeling the eyeball by determining at least one corner of the eyeball. In a number of embodiments, the center of the sphere modeling the eyeball, the radius of the sphere modeling the eyeball and an orientation of xₑ, yₑ plane relative to the imaging system are determined by determining the corners of the eye. The corners of the eye may, for example, define aye axis of the sphere modeling the eyeball.

In a number of embodiments, an initial estimate of the position of the iris on the θ, φ plane is made. The initial estimate of the position of the iris on the θ, φ plane may, for example be made using a Though Transform. A further estimation of the position of the iris on the θ, φ plane may, for example, be made using a circle fitting algorithm that is different from an algorithm used in the initial estimate.

Gaze may, for example, be estimated using a model that relates the position of the iris on the θ, φ plane to the gaze.

The method may, for example, further include imaging a second eyeball using the imaging system over time using light in the visible spectrum, modeling the second eyeball as a sphere, determining the position of the iris of the second eyeball on the sphere modeling the second eyeball by (i) mapping an image from the imaging system to a θ′, φ′ plane wherein θ′ is the polar angle about an axis zₑ′ emanating from the center of the sphere modeling the second eyeball, φ′ is the azimuth angle in an xₑ′, yₑ,′ plane which passes through the center of the sphere modeling the second eyeball and is orthogonal to the zₑ axis, and (iii) detecting the circle modeling the iris of the second eyeball on the θ′, φ′ plane, and estimating the direction of the subject's gaze from the determined position of the iris on the eyeball and the second eyeball.

In a number of embodiments, only light in the visible spectrum is used to image the eyeball. Only ambient light in the visible spectrum may, for example, used to image the eyeball. A light source may be provided in a number of embodiments.

The method may further include tracking a position of the eye relative to the imaging system to account for movement of the position of the eye relative to the imaging system in determining the subject's gaze. A position of the corners of the eye may, for example, be used to determine a position of the eye relative to the imaging system.

The method may further include providing an outside imaging system in operative connection with the headgear, imaging an outside environment using an outside imaging system over time, and relating the subject's gaze to an image of the outside environment. The method may further include calibrating by having the subject gaze at an objecting in the outside environment having a known position.

In another aspect, a method for estimating a direction of a subject's gaze includes providing headgear to be worn by the subject, the headgear including an imaging system in operative connection with the headgear, imaging at least one eyeball using the imaging system over time, determining the position of the iris on the eyeball by modeling the eyeball as a sphere and modeling the iris as a circle movable on the sphere modeling the eyeball, and estimating the direction of the subject's gaze from the determined position of the iris on the eyeball. Further aspects of the method may, for example, be as described above.

In another aspect, a system for estimating a direction of a subject's gaze includes headgear to be worn by the subject, an imaging system in operating connection with the headgear, the imaging system being adapted to image at least one eyeball over time using light in the visible spectrum, an outside imaging system adapted to image an outside environment viewed by the subject, and a processing system adapted to determine the position of the iris on a model of the eyeball and estimate the direction of the subject's gaze from the determined position of the iris on the model of the eyeball.

In a further aspect, a system for estimating a direction of a subject's gaze includes headgear to be worn by the subject, an imaging system in operative connection with the headgear, the imaging system being adapted to image at least one eyeball over time, an outside imaging system adapted to image an outside environment viewed by the subject, and a processing system adapted to (i) determine the position of the iris on a sphere modeling the eyeball by mapping an image from the imaging system to a θ, φ plane wherein θ is a polar angle about an axis zₑ emanating from a center of the sphere modeling the eyeball, φ is an azimuth angle in an xₑ, yₑ plane which passes through the center of the sphere modeling the eyeball and is orthogonal to the zₑ axis, and detecting a circle modeling the iris on the θ, φ plane and (iii) to estimate the direction of the subject's gaze from the determined position of the iris on the sphere modeling the eyeball.

In still a further aspect, a system for estimating a direction of a subject's gaze includes headgear to be worn by the subject, an imaging system in operative connection with the headgear, the imaging system being adapted to image at least one eyeball over time, an outside imaging system adapted to image an outside environment viewed by the subject, and a processing system adapted (i) to determine the position of the iris on a sphere modeling the eyeball, wherein the iris is modeled as a circle movable on the sphere modeling the eyeball and (iii) to estimate the direction of the subject's gaze from the determined position of the iris on the eyeball.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a schematic of the eye model used in the present invention.

FIG. 2 illustrates schematically the rotation between the by camera axes and the xₑyₑ eyeball axes.

FIG. 3 illustrates schematically parameterization of the surface of the eyeball sphere.

FIG. 4 illustrates schematically representations of the iris on eye images (top) and the (θ,φ) plane (bottom) for multiple values of the eye center (θ_{I},φ_{I}).

FIG. 5A illustrates an original eye image I (u,v).

FIG. 5B illustrates pixels corresponding to the (θ,φ) plane

FIG. 5C illustrates an “unwrapped” image I (θ,φ) resembled from the original image of FIG. 5A.

FIG. 6A illustrates voting procedures for a Though Transform wherein the points with a gradient above a threshold are the only ones to vote.

FIG. 6B illustrates that each point votes for multiple locations of the center of the iris along the opposite direction of its gradient.

FIG. 7 illustrates schematically a search for features proceeding along paths radiating from a current estimate of the center of the iris (θ_{I},φ_{I}).

FIG. 8 illustrates schematically an example of an estimated circle as well as inkier and outliner features.

FIG. 9 illustrates detected iris edge points overlaid on the original input image.

FIG. 10 illustrates eye image and corresponding out images obtained for multiple locations of a 25 target screen, which were used in some embodiments to calibrate the gaze model.

FIG. 11A illustrates an image of an eye taken in calibration with the eye model superimposed.

FIG. 11B illustrates an unwrapped image corresponding to the image of FIG. 11A.

FIG. 11C illustrates another image of an eye with the eye model superimposed after the position of the camera had changed relative to the position of the eye.

FIG. 11D illustrates an unwrapped image corresponding to the image of FIG. 11B, which demonstrates incorrect modeling of the eye resulting in distortion as a result of the position of the camera changing relative to the position of the eye.

FIG. 12A illustrates side schematic view of a subject wearing headgear (for example, a helmet) with an indication of the manner in which an imaging system (for example, including one or more cameras), which is in operative connection with the headgear, may move relative to the position of the eye.

FIG. 12B illustrates a front schematic view of a subject wearing a helmet including a system hereof

FIG. 13A sets forth the percentage of correct eye corner detections determined as the number of times that the detection is within 5% of the eye radius from the ground truth divided by the total number of analyzed frames for one embodiment of a system hereof

FIG. 13B illustrates the time to compute the best match for correct eye corner detections for one embodiment of a system hereof

FIG. 14A illustrates a percentage of frames where the distance from the detected eye center to the ground truth is below a given normalized distance (that is, the distance divided by the eye radius) wherein previous history is not used to stabilize the results for one embodiment of a system hereof

FIG. 14B illustrates a percentage of frames where the distance from the detected eye center to the ground truth is below a given normalized distance (that is, the distance divided by the eye radius) wherein previous history is used to stabilize the results for one embodiment of a system hereof

FIG. 15A illustrates the image of an eye (an “unwrapped” image of the eye surface) corresponding to FIG. 11C after the position of the camera had changed relative to the position of the eye.

FIG. 15B illustrates an unwrapped image corresponding to the image of FIG. 15A, and demonstrates correct modeling of the eye upon accounting for the position of the camera changing relative to the position of the eye.

FIG. 16A illustrate an example of output from the system of FIG. 12B.

FIG. 16B illustrate another example of output from the system of FIG. 12B.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a camera” includes a plurality of such cameras and equivalents thereof known to those skilled in the art, and so forth, and reference to “the camera” is a reference to one or more such cameras and equivalents thereof known to those skilled in the art, and so forth.

In a number of embodiments, systems and methods for estimating the direction of a subject's gaze are described. In a number of embodiments, systems and methods hereof use light in the visible spectrum to determine the position of the iris on a model of the eyeball. In a number of embodiments, methods and systems hereof may alternatively or additionally be used connection with light outside the visible spectrum (for example, infrared light). In a number of such embodiments, only ambient light is used (that is, the systems and methods hereof do not include a dedicated light, source but use light from the surrounding environment to determine the position of the iris on a model of the eyeball). In circumstances in which ambient light is insufficient for imaging, a source of light may be used. At least one parameter of the model of the eyeball may, for example, be determined by determining the position of at least one corner (and typically both corners) of the eye. The position of the iris on a model of the eyeball may, for example, be determined by detecting at least a portion of an edge or perimeter of a model of iris (for example, an elliptical model, a circular model etc.) On the model of the eyeball (for example, a spherical model). Determining an edge or perimeter of a model of the iris is particularly useful when light in the visible spectrum is used to determine the position of the iris on a model of the eyeball.

In a number of embodiments, systems and methods hereof represents the eyeball as a three-dimensional sphere and the iris as a two-dimensional circle on the sphere. In a number of embodiments, systems and methods hereof determine the position of the iris on a sphere modeling the eyeball by mapping an image from the imaging system to a θ, φ plane wherein θ is the polar angle about an axis zₑ emanating from the center of the sphere modeling the eyeball, 0 is the azimuth angle in an xₑ, yₑ, plane which passes through the center of the sphere modeling the eyeball and is orthogonal to the zₑ axis. The position of the iris of the sphere modeling the eyeball on the θ, φ plane may, for example, be determined by detecting a circle modeling the iris of the eyeball on the θ, φ plane.

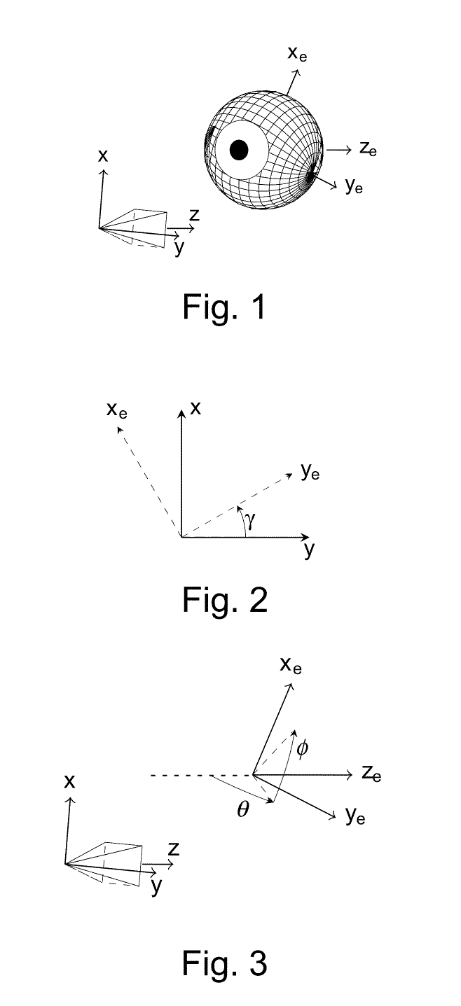

FIG. 1 illustrates an embodiment of a spherical eye model hereof X_{c}=[x_{c},y_{c},z_{c}]^{T} is defined as the coordinate of the eyeball model center in the camera coordinate system (x,y,z). On the eyeball model center in a number of embodiments, the axes (xₑ,yₑ,zₑ) are defined so that the eye plane is parallel to the image plane (i.e., zₑ is parallel to z.). The method may, for example, assume that the eye corners are on this plane and define the yₑ axis so that it is aligned with the eye corners. Because, in a number of embodiments, the systems and methods hereof are used with wearable devices (for example, glasses, goggles or helmets), the systems and models are first discussed herein assuming that the center the eyeball sphere is fixed relative to the position of the camera. In such embodiments, the camera and the eyeball model axes are related by a rotation about the z axis, plus a translation from the center of the camera to the center of the eye. The angle y is defined as be the rotation along the z axes between the camera and the eye axes as denoted in FIG. 2. For clarity, the figure assumes no translation between axes. The z axis is collinear to zₑ with both axes pointing into the paper in FIG. 2. The relationship between the camera and the eyeball axes is:

$$[ \begin{matrix}x \\ y \\ z\end{matrix} ] = R ( - γ ) [ \begin{matrix}{x}_{e} \\ {y}_{e} \\ {z}_{e}\end{matrix} ] + {X}_{C} ⇔ [ \begin{matrix}x \\ y \\ z\end{matrix} ] = [ \begin{matrix}cos γ & - sin γ & 0 \\ sin γ & cos γ & 0 \\ 0 & 0 & 1\end{matrix} ] [ \begin{matrix}{x}_{e} \\ {y}_{e} \\ {z}_{e}\end{matrix} ] + [ \begin{matrix}{x}_{C} \\ {y}_{C} \\ {z}_{C}\end{matrix} ] \qquad ( 1 )$$

Parameterization of the sphere surface is illustrated in FIG. 3. For clarity, the eyeball sphere (model) is not shown in FIG. 3. The angles (θ,φ) parameterize the surface of the eyeball sphere model. Assuming that the radius of the sphere is r, the coordinates on the eyeball axes of a point in the surface of the of the eyeball sphere are:

$$[ \begin{matrix}{x}_{e} \\ {y}_{e} \\ {z}_{e}\end{matrix} ] = {R}_{y} ( φ ) {R}_{x} ( θ ) [ \begin{matrix}0 \\ 0 \\ - r\end{matrix} ] ⇔ [ \begin{matrix}{x}_{e} \\ {y}_{e} \\ {z}_{e}\end{matrix} ] = [ \begin{matrix}cos φ & 0 & - sin φ \\ 0 & 1 & 0 \\ sin φ & 0 & cos φ\end{matrix} ] [ \begin{matrix}1 & 0 & 0 \\ 0 & cos θ & - sin θ \\ 0 & sin θ & cos θ\end{matrix} ] [ \begin{matrix}0 \\ 0 \\ - r\end{matrix} ] \qquad ( 2 )$$

The same point will have the following coordinates on the camera axes:

$$[ \begin{matrix}x \\ y \\ z\end{matrix} ] = {R}_{z} ( - γ ) {R}_{y} ( φ ) {R}_{x} ( θ ) [ \begin{matrix}0 \\ 0 \\ - r\end{matrix} ] + [ \begin{matrix}{x}_{C} \\ {y}_{C} \\ {z}_{C}\end{matrix} ] ⇔ \\ [ \begin{matrix}x \\ y \\ z\end{matrix} ] = R ( θ , φ , γ ) [ \begin{matrix}0 \\ 0 \\ - r\end{matrix} ] + {X}_{C}$$

wherein R(θ,φ,γ) is defined to be R_{z} (−γ)R_{y} (φ)Rₓ (θ) and the rotation matrices are defined by:

$${R}_{z} ( - γ ) = [ \begin{matrix}cos γ & - sin γ & 0 \\ sin γ & cos γ & 0 \\ 0 & 0 & 1\end{matrix} ]$$

$${R}_{y} ( φ ) = [ \begin{matrix}cos φ & 0 & sin φ \\ 0 & 1 & 0 \\ - sin φ & 0 & cos φ\end{matrix} ]$$

$${R}_{x} ( θ ) = [ \begin{matrix}1 & 0 & 0 \\ 0 & cos θ & sin θ \\ 0 & - sin θ & cos θ\end{matrix} ]$$

In a number of embodiments, the iris is modeled as a two dimensional circle on the eyeball sphere model. We assume that, while the eyeball sphere model is fixed, the iris circle can move freely over it. The center of the iris circle is denoted by (θ_{I},φ_{I}) and is assumed to be in the range (θ_{I},φ_{I}) ∈ [−^{π}/2.^{π}/2]×[^{-π}/2,^{π}/2] whereas the radius of the iris circle on the (θ,φ) plane is denoted by r_{I}. The iris circle detection task reduces to determining the location of and radius of the iris circle on the eye sphere. The figures show the iris and the pupil for clarity of illustration, however (in a number of embodiments) the systems and methods hereof are only concerned with detecting the iris location.

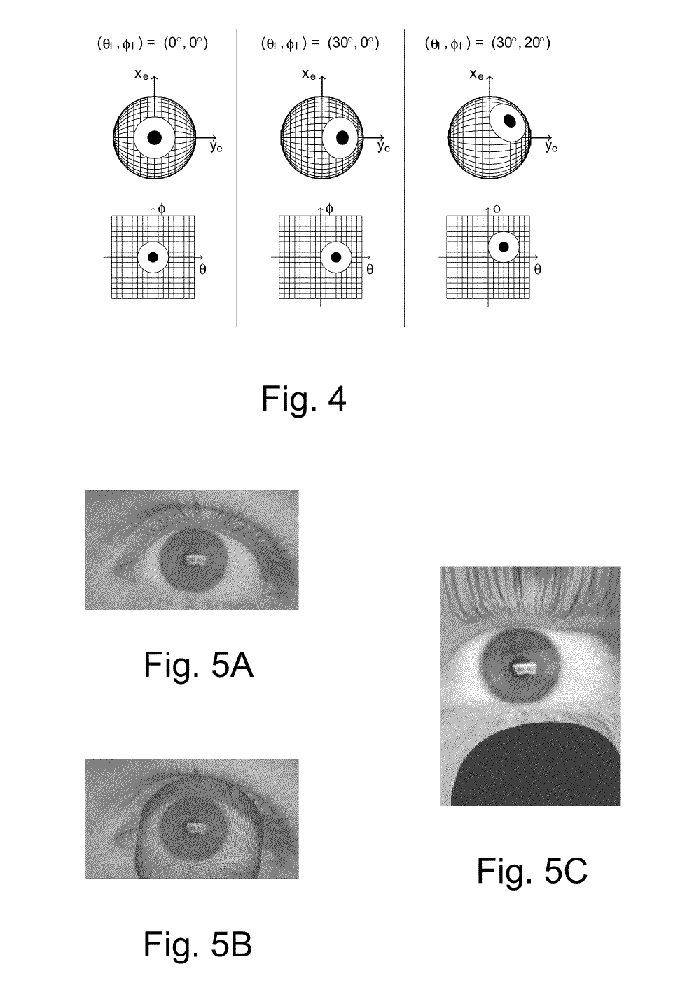

The top portion of FIG. 4 illustrates the eye model for multiple values of (θ_{I},φᵢ). For clarity, the eyeball model axis (xₑ,yₑ,zₑ) in the figure are aligned so that zₑ points into the paper in FIG. 4. As the eye moves and the iris location changes, its appearance on the image changes from that of a perfect circle to an ellipse. This makes it difficult to detect and track.

The bottom portion of FIG. 4 illustrates, for the same values of (θ_{I},φ_{I}), the iris appearance on the (θ,φ) plane. Each gradation on the θ and φ axes represents ten degrees. As the figure illustrates, although the iris may appear to be an ellipse on the image of the eye, the iris maintains its circular shape on the (θ,φ) plane. In other words, although the iris apparent shape changes on the image plane, it does not change on the unwrapped (θ,φ) plane. This observation underlines one approach to the iris detection problem, which we refer to as “unwrapping” the eye. Instead of detecting the iris in the image plane, we detect the iris in the unwrapped (θ,φ) plane.

Two sets of eye model parameters may be defined. First, the parameters that describe the eye sphere: the rotation γ with relation to the camera, the center of the eyeball (x_{c},y_{c},z_{c}), and the radius of the eyeball r. And second, the parameters that describe the iris on the unwrapped (θ,φ) plane, namely its location (θ_{I},φ_{I}) and radius r_{I}. In a number of embodiments, the systems and methods hereof are calibrated by having the subject gaze at one or more items or objects having a known position. Objectives of the calibration of the eye model include determining the eye sphere parameters (namely, the γ angle, and the center and radius of the eyeball) which are assumed fixed.

A number of embodiments of calibration methods hereof require only the knowledge of, for example, the location of the two eye corners in the image, denoted as (u_{L}, v_{L}) and (u_{R}, v_{R}). These locations may, for example, be obtained manually (by asking the subject or user to click the eye corners,) or automatically (by, for example, using descriptive features tailored for the detection of the eye corners). In the eyeball model coordinate system the eye corners have coordinates (0,±r,0).

Assuming that the intrinsic parameters of the eye camera are known and letting K be the invertible matrix of camera parameters, one has the following relationship between the location of the corners in the image and in the eyeball coordinate system (where we assume the standard perspective camera model, and use (u_{DR},v_{DR}) to denote either the left or right corners):

$$[ \begin{matrix}{u}_{LR} \\ {v}_{LR} \\ 1\end{matrix} ] = λ K [ \begin{matrix}cos γ & - sin γ & 0 \\ sin γ & cos γ & 0 \\ 0 & 0 & 1\end{matrix} \vert \begin{matrix}{x}_{C} \\ {y}_{C} \\ {z}_{C }\end{matrix} ] [ \begin{matrix}0 \\ \pm r \\ 0 \\ 1\end{matrix} ] , λ \neq 0 \qquad ( 4 )$$

Defining (u′_{DR},v′_{DR}) to be such that:

$$[ \begin{matrix}{u}_{LR}^{′} \\ {v}_{LR}^{′} \\ 1\end{matrix} ] = {K}^{- 1} [ \begin{matrix}{u}_{LR} \\ {v}_{LR} \\ 1\end{matrix} ] \qquad ( 5 )$$

one can write:

$$[ \begin{matrix}{u}_{LR}^{′} \\ {v}_{LR}^{′} \\ 1\end{matrix} ] = λ [ \begin{matrix}{x}_{C} ∓ sin γ \\ {y}_{C} \pm cos γ \\ {z}_{C}\end{matrix} ] , λ \neq 0 \qquad ( 6 )$$

which allows one to find the following relationships:

$${x}_{C} = \frac{1}{2 λ} ( {u}_{L}^{′} + {u}_{R}^{′} ) \\ {y}_{C} = \frac{1}{2 λ} ( {u}_{L}^{′} + {v}_{R}^{′} ) \\ {z}_{C} = \frac{1}{λ} \\ r = \frac{1}{2 λ} \sqrt{{( {u}_{L}^{′} - {u}_{R}^{′} )}^{2} + {( {v}_{L}^{′} - {v}_{R}^{′} )}^{2}} \\ γ = \{ \begin{matrix}arctan ( - \frac{{u}_{L}^{′} - {u}_{R}^{′}}{{v}_{L}^{′} - {v}_{R}^{′}} ) & {v}_{L}^{′} \neq {v}_{R}^{′} \\ 0 & {v}_{L}^{′} = {v}_{R}^{′}\end{matrix} \qquad ( 7 )$$

Thus, simply knowing the eye corners' location in the image is sufficient to fully determine y and to determine the remaining fixed parameters up to a constant scaling factor. These parameters are then used to determine, in real time, the iris parameters.

After the eye model is calibrated, the location where each point on the (θ,φ) plane lies in the eye image is determined by using the standard perspective camera model and Equation (3):

$$[ \begin{matrix}u ( θ , φ ) \\ v ( θ , φ ) \\ 1\end{matrix} ] = λ K ( R ( θ , φ , γ ) [ \begin{matrix}0 \\ 0 \\ - r\end{matrix} ] + {X}_{C} ) , λ \neq 0 \qquad ( 8 )$$

This allows the method to re-sample the eye image to obtain the pixel values corresponding to the (θ,φ) plane and create an image similar to FIG. 4. If the original image is I (u,v), by using Equation (8), an image I (θ,φ) is created such that:

I(θ,φ)=I(u(θ,φ),v(θ,φ)) ∀(θ,φ) ∈[^{-π}/2,^{π}/2]×[^{-π}/2,^{π}/2] (9)

FIGS. 5A through 5C presents an example of a I (θ,φ) image created by an embodiment of a system hereof. FIG. 5A illustrates the original eye image I (u,v), while FIG. 5C illustrates pixels corresponding to the (θ,φ) plane. FIG. 5C illustrates the “unwrapped” image I (θ,φ) resembled from the original image. On the “unwrapped” image, the iris has a circular shape, the eyelids are approximately horizontal and the eyelashes are approximately vertical.

As discussed above, the iris has the shape of a circle on the (θ,φ) plane, regardless of the gaze direction. Thus, we model the iris as a circle with three parameters, the center (θ_{I},φ_{I}) and a radius

(θ,φ) ∈ iris [symbol] (θ−θ_{I})²+(φ−φ_{I})²≦rI² (10)

In theory, the iris radius r_{I} is constant for every frame in the eye video and could be determined during the calibration of the eye model. In practice, however, we found that, because the eyeball is not a perfect sphere and because there can be small errors in the eye calibration, the apparent radius of the iris in the (θ,φ) plane is not constant. Therefore the method does not attempt to estimate a fixed value for R_{I} and instead allows it to vary within a small range r_{I} ∈ R_{I}. In several embodiments, R_{I} was set to allow for a variation of +/−5 degrees. The limits on the allowed variation may readily be established or optimized for a particular device or system.

In a number of embodiments, the Though Transform (an estimation technique used in, for example, image analysis) was used to estimate the model parameters through a voting procedure. In the voting procedure, each observation votes for the values of the parameters that agree with it. In a number of embodiments, the method uses the Though Transform to allow each point on the iris circumference to vote for the iris radii and centers that best agree with it.

The method, for example, starts by computing the gradient of a Gaussian smoothed version of I (θ,φ), which we denote by G(θ,φ). We let |G(θ,φ)| be the magnitude of the gradient and Ψ(θ,φ) be its orientation. Each point that has a gradient above a threshold votes for the radii and center along the direction to its gradient. For example, if |G(θ⁰,φ⁰)|>Gₜₕᵣ then the point (θ⁰,φ⁰) votes for the following θ_{I},φ_{I},r_{I} combinations:

$$∀ {r}_{I}^{′} ∈ {R}_{I} \text{:} vote for \{ \begin{matrix}{φ}_{I} = {θ}^{′} - {r}_{I}^{′} cos [ Ψ ( θ , φ ) ] \\ {φ}_{I} = {φ}^{′} - {r}_{I}^{′} sin [ Φ ( θ , φ ) ] \\ {r}_{I} = {r}_{I}^{′}\end{matrix} \qquad ( 11 )$$

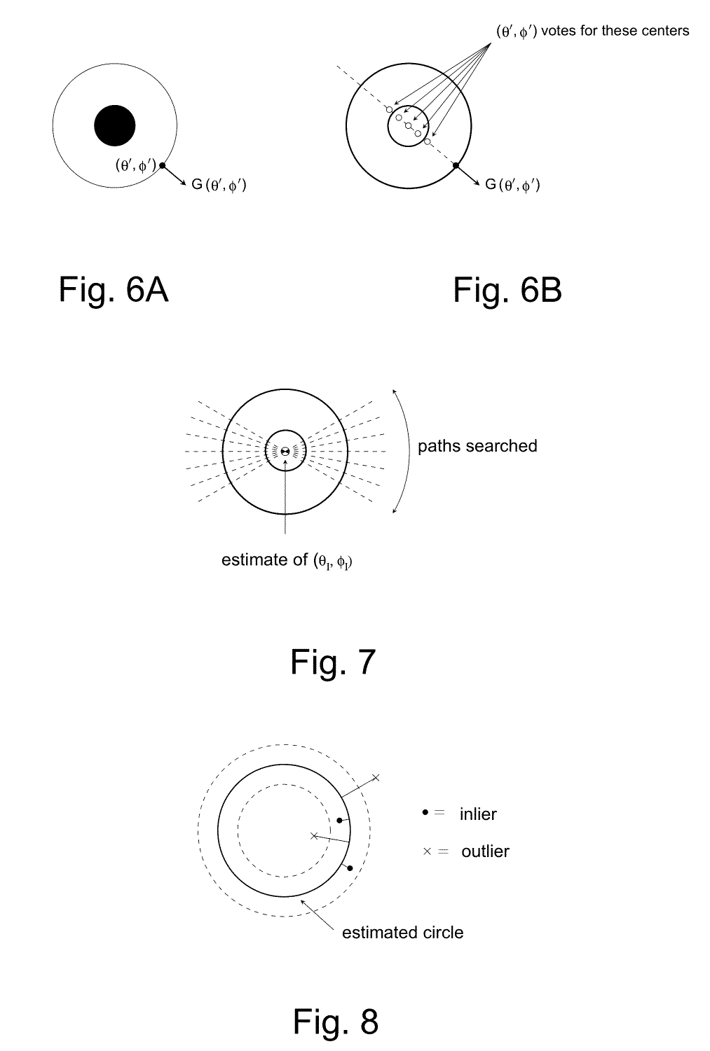

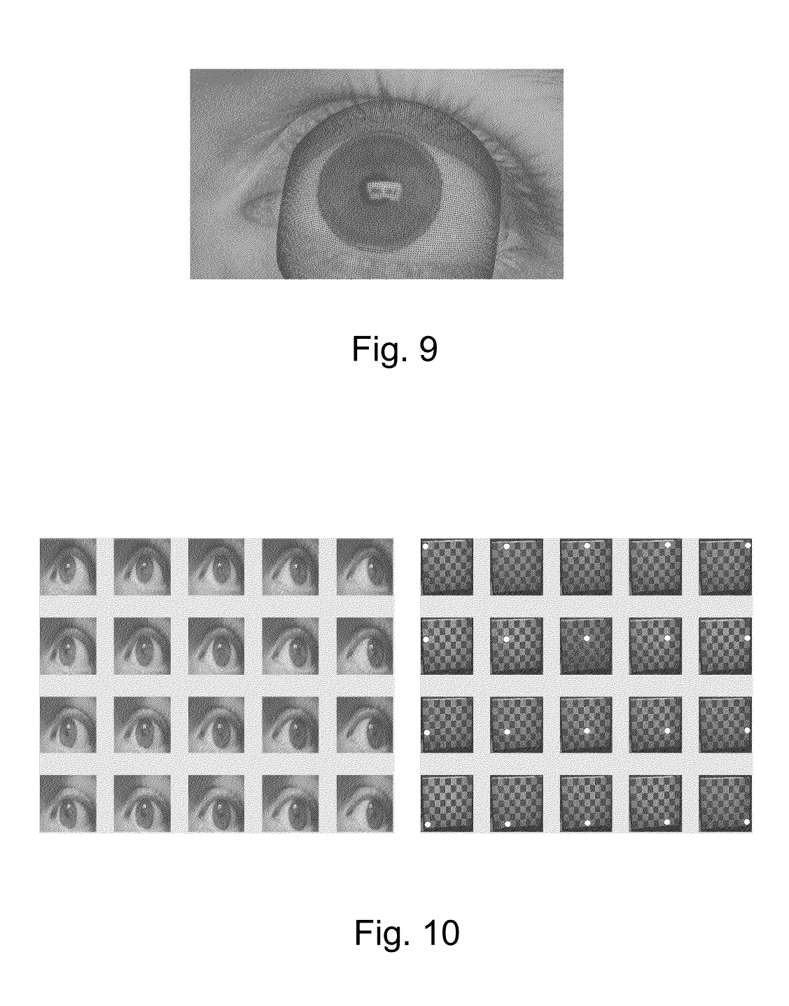

FIGS. 6A and 6B illustrate this voting procedure. FIG. 6A illustrates that the points with gradient above a threshold are the only ones to vote. FIG. 6B illustrates that each point votes for multiple locations of the center of the iris along the opposite direction of its gradient. After all the points vote, the votes are tallied into bins with each vote also counting towards its neighbors in the θ_{I},φ_{I}, and r_{I} directions. The initial estimate of the circle parameters corresponds to the bin with the highest number of votes.

Based on the initial estimate of the circle location, the method detects features of the iris circle. Starting at the estimate of the circle center, it samples I (θ,φ) along paths owing outwards of the center as illustrated in FIG. 7. In a number of embodiments, the method considers only paths close to the horizontal direction to minimize the interference of the eyelids on the feature detection process. To detect features, the method searches for the maximum of the gradient along each of these paths.

The method may, for example, use an algorithm proposed by G. Taubin to estimate the circle that best fits the features detected in the previous step. See Taubin, G., “Estimation Of Planar Curves, Surfaces And Nonplanar Space Curves Defined By Implicit Equations, With Applications To Edge And Range Image Segmentation”, IEEE Trans. PAMI, Vol. 13, pages 1115-1138, (1991), the disclosure of which is incorporated herein by reference. This method produces accurate circle fits even if data points are observed only within a small arc, but is sensitive to the presence of outliers.

The method may, for example, use Random Sample Consensus or RANSAC to make this algorithm robust to the presence of outliers. In particular, it randomly selects small groups of features. For each group, the method may, for example, use Taubin's method to estimate the center (θ_{I},φ_{I}) and radius r_{I} of the circle and select the estimate of parameters that produces the most number of inliers. To count the number of inliers for each parameter estimate, the method thresholds the distance of each point to the circle as illustrated in FIG. 8 (which illustrates the estimated circle as well as the inkier and outliner features).

Once the method estimates the center of the iris circle in the (θ,φ) plane, we use Equation (3) to determine the location of the iris on the eye image, which we denote by (w,w), as well as the pixels corresponding to the detected circle.

$$[ \begin{matrix}{u}_{G} \\ {v}_{G} \\ 1\end{matrix} ] = [ \begin{matrix}{h}_{11} & {h}_{12} & {h}_{13} \\ {h}_{21} & {h}_{22} & {h}_{23} \\ {h}_{31} & {h}_{32} & h 33\end{matrix} ] [ \begin{matrix}{u}_{I} \\ {v}_{I} \\ 1\end{matrix} ] \qquad ( 12 )$$

To determine the parameters of the relationship, H, the gaze model is calibrated by presenting the user with a target screen as shown on FIG. 10. In a number of embodiments, the user is asked to click on a circle or other object of known position in a display screen multiple times in different locations on the display screen. The process may be repeated multiple times with the target circle in different positions as illustrated in FIG. 10.

Each time t ∈{1, . . . ,T} that the user clicks on the circle, the system determines the iris location on the eye image (u_{I}^{t}:v_{I}^{t}), as well as the circle location on an outside or outward image (u_{C}^{t}, v_{C}^{t}). By assuming that the user is looking at the target when it clicks on it, this procedure determines T relationships between the iris and the gaze locations. Based on this relationships, the system uses RANSAC to determine the parameters, H, of the gaze model.

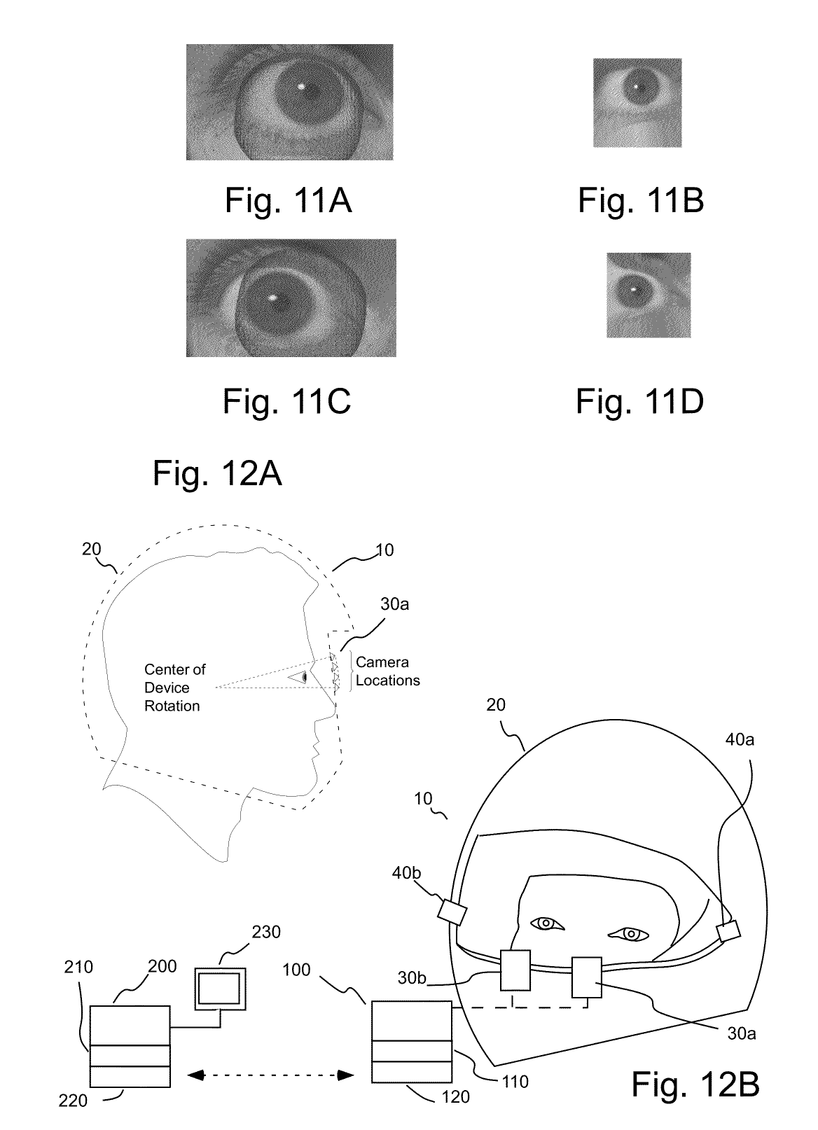

The method described above works well for stable applications, such as when the user is sitting and gazing at a screen. However, distortions and/or inaccuracies may be introduced in situations where the user's activities may cause the imaging system/camera(s) to move with relationship to the head (and consequently) the eye of the wearer. FIGS. 11A through 11D provide an example. FIGS. 11A and 11B set forth the original image and the unwrapped image, respectively from a calibration procedure. FIG. 11C and 11D illustrate distortion that results in the unwrapped image (FIG. 11D) resulting from movement of the camera relative to the eye. By tracking the position of the eye (or eyes) relative to the position of the imaging system/camera(s), distortions arising from camera movement relative to eye can be reduced or eliminated. For example, the eye corner(s) (or other distinctive features visible in the camera feed and annotated by the operator or determined automatically) may tracked and the 3-D eye model adjusted from frame to frame. This procedure allows the systems and methods hereof to obtain a consistent unwrapped eye surface image and thus achieve accurate iris detection.

Although the eye location, when observed from the eye camera, can change significantly, the distortions are limited by the geometry of the wearable device used. For example, for cameras mounted on helmets or eyeglasses, the motion corresponds to a rotation centered on the head of the user (see, for example, FIG. 12A and 12B). Because the distance from the eye to the camera remains approximately constant and because the radius of the camera rotation is much larger than the distance to the eye, the movement distortions in the eye image may, for example, be accurately modeled by a translation.

Mathematically, the argument above states that, on the camera reference coordinates (x,y,z), the movement of the eye center Xi with regards to the eye camera can be modeled by:

$${X}_{c} = [ \begin{matrix}{x}_{c} \\ {y}_{c} \\ {z}_{c}\end{matrix} ] = [ \begin{matrix}{x}_{c}^{0} + {x}_{c} ( t ) \\ {y}_{c}^{0} + {y}_{c} ( t ) \\ {z}_{c}^{0}\end{matrix} ] \qquad ( 13 )$$

wherein

X_{c}=[x_{c}⁰y_{c}⁰z_{c}⁰]

wherein X_{c}=[x_{c}⁰y_{c}⁰z_{c}⁰] corresponds to the original eye center location determined during calibration and the movement is modeled by the terms x_{c}(t) and y_{c}(t).

Using equation 1, and assuming an upper triangular intrinsic camera matrix, equation 13 implies that locations of the corners of the eye can be modeled by:

$$\{ \begin{matrix}{u}_{l} = {u}_{l}^{0} + u ( t ) \\ {u}_{r} = {u}_{r}^{0} + u ( t )\end{matrix} \\ \{ \begin{matrix}{v}_{l} = {v}_{l}^{0} + v ( t ) \\ {v}_{r} = {v}_{r}^{0} + v ( t )\end{matrix} \qquad ( 14 )$$

which shows that the camera movement is modeled by a translation in the eye image with time varying parameters u(t) and v(t).

During calibration, in addition to highlighting the user's eye corners, the operator may, for example, select a range of expected translations to be observed as a result of the movements of the user's head in relation to the wearable device (and the imaging system/camera(s) attached thereto). The eye corner information may, for example, be used to select a region of the image as a template for each eye corner Tₗ(u,v) and Tᵣ(u,v). At run-time, template matching may, for example, be used to determine the correct location of the eye corners.

Template matching works by comparing all segments in the region of interest selected by the operator to the corresponding eye corner template, and determining the best match. For the left corner, we have:

$$( {\hat{u}}_{l} , {\hat{v}}_{l} ) = arg {max}_{( {u}_{l} , {v}_{l} )} S ( I ( u + {u}_{l} , v + {v}_{l} ) , {T}_{l} ( u , v ) ) \qquad ( 15 )$$

where the optimization is done for every eye image but the time index t was omitted for compactness, and S is a similarity measure between the two image segments. The result for the right corner is equivalent.

In several studies, to determine the best measure of template similarity S [symbol] (u+uₗ, v+vₗ), [symbol] (u, v)) we compared four different measures (all summations are done over the range of (u,v) values where the template was sampled) as follows:

Squared Difference (sqdiff):

$$S ( \cdot ) = - {\sum }_{u , v} {[ I ( u + {u}_{l} , v + {v}_{l} ) - {T}_{l} ( u , v ) ]}^{2} \qquad ( 16 )$$

Normed Squared Difference (sqdiffNormed):

$$S ( \cdot ) = - \frac{{\sum }_{u , v} {[ I ( u + {u}_{l} , v + {v}_{l} ) - {T}_{l} ( u , v ) ]}^{2}}{\sqrt{{\sum }_{u , v} {I}^{2} ( u + {u}_{l} , v + {v}_{l} ) \cdot {\sum }_{u , v} {T}_{l}^{2} ( u , v )}} \qquad ( 17 )$$

Normed Cross Correlation (ccorrNormed):

$$S ( \cdot ) = \frac{{\sum }_{u , v} [ I ( u + {u}_{l} , v + {v}_{l} ) \cdot {T}_{l} ( u , v ) ]}{\sqrt{{\sum }_{u , v} {I}^{2} ( u + {u}_{l} , v + {v}_{l} ) \cdot {\sum }_{u , v} {T}_{l}^{2} ( u , v )}} \qquad ( 18 )$$

Normed Pearson Correlation (ccoefNormed): where the similarity is as in equation (18) but the image [symbol] and template [symbol] are replaced by their mean subtracted counterparts [symbol] = [symbol] − [symbol] and [symbol] = [symbol] − [symbol] . See, for example, Bradski, G., The OpenCV Library, Dr. Dobbs's Journal of Software Tools, 2000, the disclosure of which is incorporated herein by reference.

The comparison of similarity measures was performed on a dataset of 250 frames extracted from a video recorded with a helmet equipped with visible-spectrum miniature cameras. The helmet was moved with relationship to the user's eye to simulate natural conditions during the practice of, for example, a sport such as auto racing. The template was extracted from the initial frame manually, and the similarity measures were compared with the ground truth for all other frames.

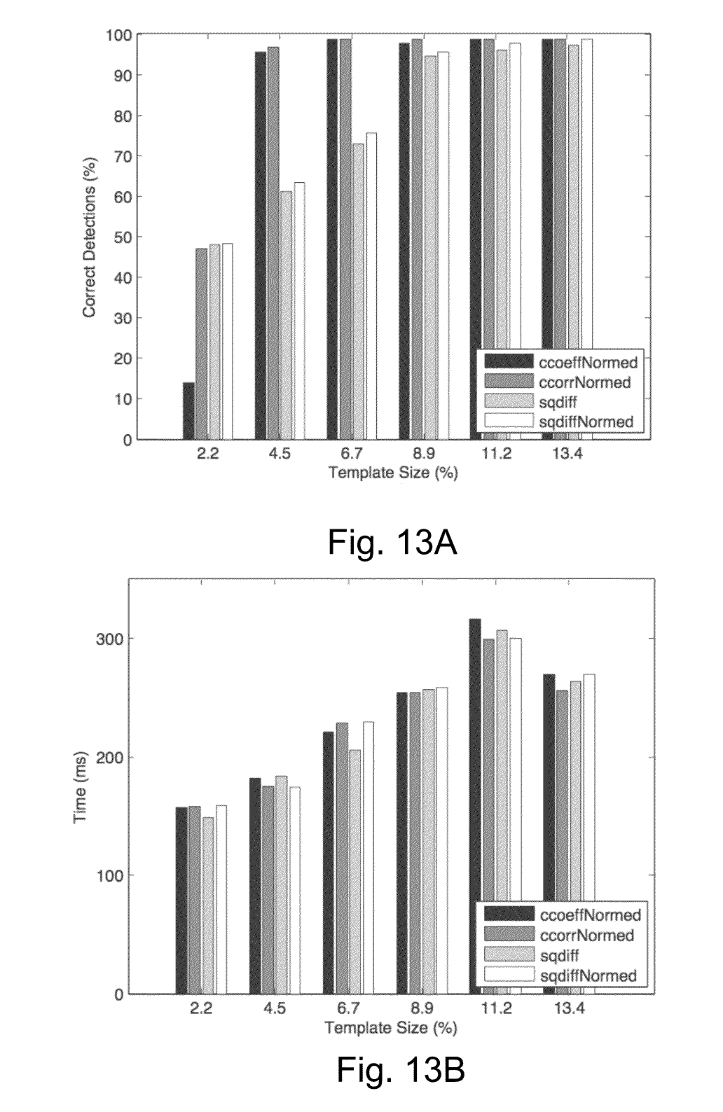

FIGS. 13A and 13B presents the percentage of correct eye corner detections (number of times that the detection is within 5% of the eye radius from the ground truth divided by the total number of analyzed frames) and the time to compute the best match, respectively, for one embodiment of a system hereof. In both cases, results are presented as a function of the template size (as a percentage of the eye radius.)

The best similarity measure and template size strike a balance between fast computation times and large percentage of correct detections. From FIGS. 13A and 13B, we determined that the Normed Cross Correlation measure with a template size of approximately 4.5% of the eye radius achieves the best trade-off

The complete iris detection method was implemented in C++ and uses OpenCV for some of the basic vision tasks. Bradski, G., The OpenCV Library, Dr. Dobbs's Journal of Software Tools, 2000, the disclosure of which is incorporated herein by reference. In practical applications the template matching task runs in parallel with the iris detection so as to not reduce the frame rate (from FIG. 13B, we know that the template matching tasks takes less than 200 ms, that is, runs at approximately 5 frames per second.) After each template matching operation the eye model is recomputed and the iris detection procedure is updated appropriately.

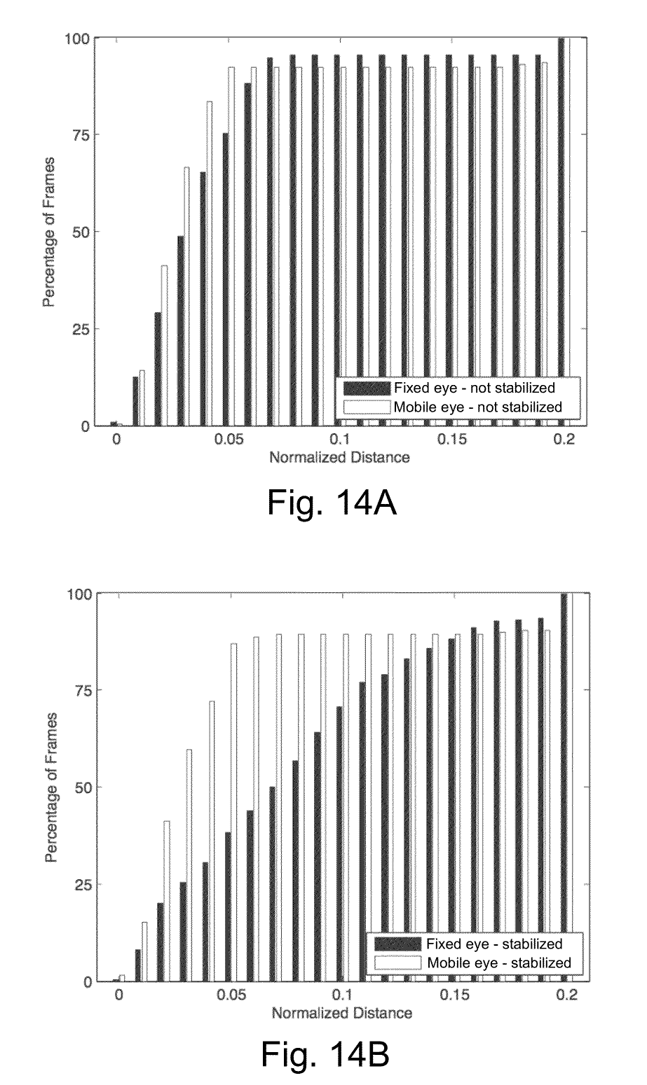

To evaluate the performance of the above described methodology which accounts for camera movement relative to the eye, we compared it against methods (in which there is no accounting for camera movement) on a different set of 250 frames (again, taken with a helmet wearable device, simulating the head movements with regard to the helmet) for one embodiment of a system hereof. To fully explore the properties of the proposed method, the corners were detected on every frame at the expense of slower frame rate. However, the performance was very similar to the real-time system. FIGS. 14A and 14B provide a summary of the results.

To produce better results, it is often useful to use the last few frames of history to “stabilize” the iris detection result (for example, by using an exponentially decaying average filter.) Under normal circumstances, this improves the quality of the detection at the expense of a small lag when there is large eye movement. During testing we discovered that omitting this stabilizing operation improves results for methods hereof wherein the eyes is assumed to be fixed relative to the camera. This can be explained by the fact that, since this method assumes the eye is fixed, the movements of the eye ball with regard to the camera are actually interpreted as very rapid movements of the iris on a fixed eyeball. In methods in which the relative movement of eyeball and the camera are tracked, fairly constrained iris movement is observed, and omitting the stabilization step on such cases reduces (slightly) the performance.

FIGS. 14A and 14B shows that, even in the best case, the method in which output is adjusted for camera/eye relative motion always outperforms the method in which output is not adjusted to account for camera/eye relative motion . The above described methodology to account for camera/eye relative motion may still have a failure case when the distortion is so large that the corners are not detected. These cases however, may, for example, be mitigated by modeling the movement of the eye corners over time or using more advanced matching method.

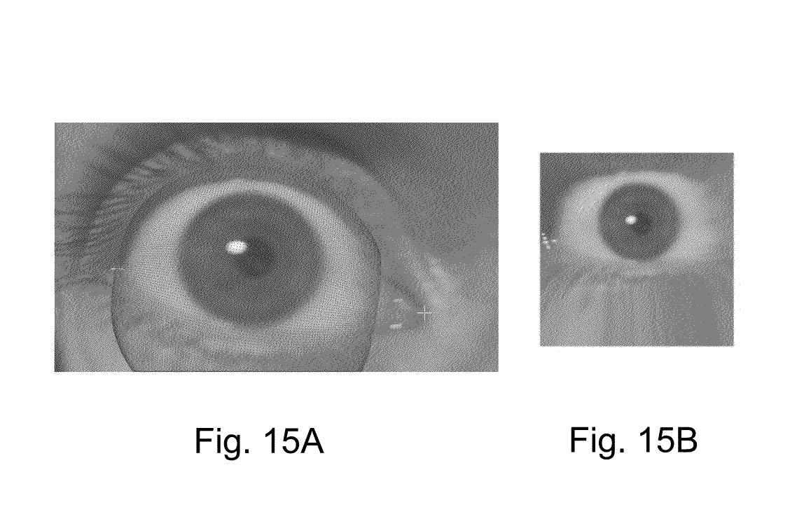

FIG. 15A and 15B illustrates the advantages of the method in which output is adjusted for camera/eye by showing its output for the images corresponding to FIGS. 11C and 11D. As illustrated in FIG. 15B the distortions present in FIG. 11D (resulting from camera/eye relative motion) are prevented in the unwrapped image of FIG. 15B.

There are many potential applications of gaze tracking technology in sports and other activities. For the technology to be widely used in sports, as an example, it is necessary to solve a set of problems specific to the sports industry. Gaze tracking based on non-active illumination (that is, using ambient light in the visible spectrum) and which accounts for movement of the user relative to a wearable tracking system/device is desirable for field applications. As described, one embodiment of a method for accounting for such movement uses template matching to track the corners of the eyes of the user and incorporates this information into the iris detection procedure. The resulting method is fast to compute and can handle large changes in the eye location. As clear to those skilled in the art, other methods of tracking movement of the user's eyes relative to a wearable tracking/imaging system may be used.

Referring again to FIGS. 12A and 12B, a system 10 hereof may, for example, include an article of headgear such as a helmet 20 to be worn by the subject/user (for example, an auto racing drive). Other type of headgear such as eyeglasses etc. may be used. In the illustrated embodiment, system 10 includes an imaging system including at least one camera 30a to image at least one eye of the user. A second camera 30b may for example be provided wherein camera 30a images the left eye of the subject and camera images the right eye of the subject in the illustrated embodiment. Performing gaze estimation for each eye facilitates determining the depth of the subject's gaze. An outward imaging system (including, for example, one or more cameras) may be included to image the environment as seen by the subject. In the illustrated embodiment, each of cameras 30a and 30b is attached to an opening or face port in helmet 20. In the illustrated embodiment, outward imaging system includes two cameras 40 and 40b which are attached to opposite sides of helmet 20 at approximately eye level. The use of two cameras 40a and 40b enables imaging of the environment over an angle range similar to the range of vision of the subject (for example, approximately 165° in a number of embodiments).

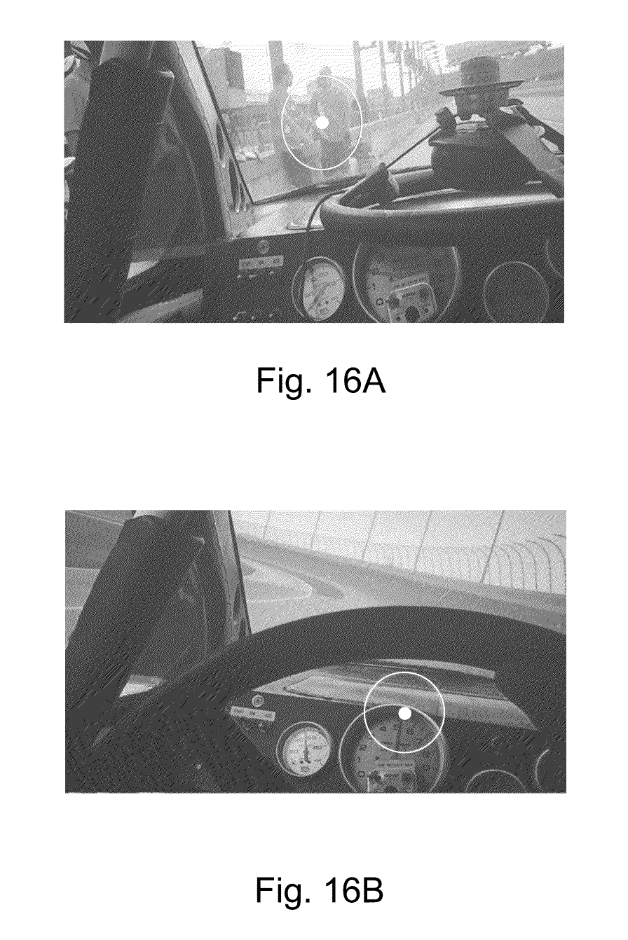

Each of cameras 30a, 30b, 40a and 40b may, for example, use light in the visual spectrum (having wavelengths from about 290 to 700 nm) to create an image. Cameras 30a, 30b, 40a and 40b may, for example, be in communicative connection with a processing system 100 (for example, including one or more microprocessors) which is adapted/programmed to carry out the algorithms described above or another algorithm to effect gaze estimation. Such algorithms may, for example, be stored in code in a memory system 110 in communicative connection with processing system 100. A communication system (including for example, one or more wireless communication modules) may also be in communicative connection with processing system 100 and memory system 110 to, for example, communicate with a remote system. Processing system 100, memory system 110 and communication system 120 may, for example, be incorporated within helmet 20 or other headgear to be worn by a subject. FIGS. 16A and 16B illustrate output from system 10 wherein the gaze estimation determined using the algorithms described above is set forth on an image of the environment viewed by the subject created by outward camera 40.

As described above, “onboard” processor 100 may be programmed to carry the algorithm(s) to effect gaze estimation. In other embodiments, processor 100 may, for example, effect only storing and/or transmitting of video streams from cameras 30a, 30b, 40a and 40b, and the processing to carry out the algorithm(s) to effect gaze estimation (and/or other processing) may be carried out by one or more remote processor systems such as a processor 200. As illustrated in FIG. 12B, processor system 200 may, for example, be in operative connection with a memory system 210, a communication system 220 (for example, a wired or wireless communication system in communicative connection with communication system 120), and a display 230. Processing may also be distributed over processor 100 and/or one or more remote processors such as processor 200. Processing may occur in real time (using real time processors as known in the art) and/or after storage of the data from the video streams for some period of time.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A method for estimating a direction of a subject's gaze, comprising:
providing headgear to be worn by the subject, the headgear including an imaging system in operative connection therewith;
imaging at least one eyeball using the imaging system over time using light in the visible spectrum,
using light in the visible spectrum to determine the position of the iris on a model of the eyeball; and
estimating the direction of the subject's gaze from the determined position of the iris on the model of the eyeball.

2. The method of claim 1 wherein at least one parameter of the model of the eyeball is determined by determining at least one corner of the eye.

3. The method of claim 1 wherein the position of the iris on a model of the eyeball is determined by detecting at least a portion of an edge of the iris on the model of the eyeball.

4. The method of claim 1 wherein the iris is modeled as a circle, the eyeball is modeled as a sphere, and the iris is modeled as a circle movable over the surface of the sphere, wherein at least one parameter of the model of the eyeball is determined by determining at least one corner of the eye and wherein the position of the iris on a model of the eyeball is determined by detecting at least a portion of an edge of the iris on the model of the eyeball.

5. The method of claim 4 further comprising mapping an image from the imaging system to a θ, φ plane wherein θ is a polar angle about an axis zₑ emanating from the center of the sphere modeling the eyeball, φ is an azimuth angle in an xₑ, yₑ plane which passes through the center of the sphere modeling the eyeball and is orthogonal to the zₑ axis, and determining the position of the iris on the θ, φ plane by detection of the circle modeling the iris on the θ, φ plane.

6. The method of claim 5 wherein center of the sphere modeling the eyeball, the radius of the sphere modeling the eyeball and an orientation of xₑ, yₑ plane relative to the imaging system are determined by determining the corners of the eye.

7. The method of claim 6 wherein the corners of the eye define aye axis of the sphere modeling the eyeball.

8. The method of claim 6 wherein the imaging system comprises a camera sensitive to light in the visual spectrum.

9. The method of claim 5 wherein an initial estimate of the position of the iris on the θ, φ plane is made.

10. The method of claim 9 wherein the initial estimate of the position of the iris on the θ, φ plane is made using a Though Transform.

11. The method of claim 9 wherein a further estimation of the position of the iris on the θ, φ plane is made using a circle fitting algorithm that is different from an algorithm used in the initial estimate.

12. The method of claim 4 wherein gaze is estimated using a model that relates the position of the iris on the θ, φ plane to the gaze.

13. The method of claim 4 further comprising:
imaging a second eyeball using the imaging system over time using light in the visible spectrum,
determining the position of the iris of the second eyeball on a sphere modeling the second eyeball by mapping an image from the imaging system to a θ′, φ′ plane wherein θ′ is the polar angle about an axis zₑ′ emanating from the center of the sphere modeling the second eyeball, φ′ is the azimuth angle in an xₑ′, yₑ, ′ plane which passes through the center of the sphere modeling the second eyeball and is orthogonal to the zₑ′ axis, and detecting a circle modeling the iris of the second eyeball on the θ′, φ′ plane; and
estimating the direction of the subject's gaze from the determined position of the iris on the eyeball and the second eyeball.

14. The method of claim 1 wherein only ambient light is used to image the eyeball.

15. The method of claim 1 further comprising tracking a position of the eye relative to the imaging system to account for movement of the position of the eye relative to the imaging system in determining the subject's gaze.

16. The method of claim 15 wherein a position of the corners of the eye are used to determine a position of the eye relative to the imaging system.

17. The method of claim 1 further comprising:
providing an outside imaging system in operative connection with the headgear;
imaging an outside environment using an outside imaging system over time; and
relating the subject's gaze to an image of the outside environment.

18. The method of claim 17 further comprising calibrating by having the subject gaze at an object in the outside environment having a known position.

19. The method of claim 1 further comprising adjusting the estimate of the direction of the subject's gaze for movement of the imaging system relative to the headgear.

20. The method of claim 19 further comprising tracking a position of at least one corner of the eye to determine movement of the imaging system relative to the headgear.

21. A method for estimating a direction of a subject's gaze, comprising:
providing headgear to be worn by the subject, the headgear including an imaging system in operative connection therewith;
imaging at least one eyeball using the imaging system over time, modeling the eyeball as a sphere;
determining the position of the iris on the sphere modeling the eyeball by mapping an image from the imaging system to a θ, φ plane wherein θ is a polar angle about an axis zₑ emanating from the center of the sphere modeling the eyeball, φ is an azimuth angle in an xₑ, yₑ plane which passes through the center of the sphere modeling the eyeball and is orthogonal to the zₑ axis, and determining the position of the iris on the θ, φ plane by detection of a circle modeling the iris on the θ,φ plane; and
estimating the direction of the subject's gaze from the determined position of the iris on the eyeball.

22. A method for estimating a direction of a subject's gaze, comprising:
providing headgear to be worn by the subject, the headgear including an imaging system in operative connection with the headgear;
imaging at least one eyeball using the imaging system over time,
determining the position of the iris on the eyeball by modeling the eyeball as a sphere and modeling the iris as a circle movable on the sphere modeling the eyeball; and
estimating the direction of the subject's gaze from the determined position of the iris on the eyeball.
